(19) United States
(12) Patent Application Publication
(10) Pub. No.: US 2013/0158547 A1
(43) Pub. Date: Jun. 20, 2013

(54) Fluid Skin Treatment Device

(71) Applicant: Jonathan David, Hermosa Beach, CA (US)

(72) Inventor: Jonathan David, Hermosa Beach, CA (US)

(21) Appl. No.: 13/720,983

(22) Filed: Dec. 19, 2012

Related U.S. Application Data
(60) Provisional application No. 61/577,444, filed on Dec. 19, 2011.

(51) Int. Cl. A61B 18/14; A61B 17/00

(52) U.S. Cl. CPC A61B 18/14; A61B 17/00
USPC 606/41; 606/131

(57) ABSTRACT

A device for performing skin treatments which includes a cartridge having a compartment for holding a treatment fluid and a reservoir for receiving a waste fluid, both of which are in communication with a source of vacuum pressure. The vacuum source withdraws the treatment fluid through an outlet in an applicator head and then draws used fluid into the waste reservoir. The applicator head can include an abrasive material for performing microdermabrasion and/or an anode and cathode for applying microcurrents.

### CROSS-REFERENCE TO RELATED APPLICATIONS

The present application claims the benefit of priority under 35 U.S.C. §120 to U.S. Patent Application No. 61/577,444, filed on Dec. 19, 2011, the contents and disclosure of which are hereby incorporated by reference in their entirety.

### BACKGROUND

Microdermabrasion is a process for removing dead cells from the outermost layer of the skin (the epidermis) to provide a younger and healthier looking appearance, remove wrinkles, clean out blocked pores, remove some types of undesirable skin conditions that can develop, and enhance skin tone. The process of microdermabrasion must be performed with a certain degree of accuracy, so that underlying live layers of skin tissue are not removed or damaged, but that enough dead cells are removed to give effective results.

Another treatment for skin and muscles, especially of the face, is microcurrent treatment. By the application of microcurrents to facial skin, the appearance fine lines and wrinkles can be diminished, leading to an improved texture and appearance of the skin. The applications of microcurrent may also aid in the penetration of some water-based products into the skin through iontophoresis. A variety of other skin treatments are also known to the art.

### FIGURES

FIG. 1 is a perspective view of an embodiment of the present device.

FIG. 2 is a side elevation view of the device of FIG. 1.

FIG. 3 is front elevation view of the device of FIG. 1.

FIG. 4 is an exploded view of the device of FIG. 1.

FIG. 5 is a sectional view along line 5-5 of FIG. 3.

FIG. 6 is an exploded view of the cartridge and applicator head components of the device of FIG. 1.

FIG. 7A is a front perspective view of the waste compartment of the device of FIG. 1.

FIG. 7B is a rear elevation view of the waste compartment of the device of FIG. 1.

FIG. 7C is a front elevation view of the waste compartment of the device of FIG. 1.

FIG. 7D is a sectional view of the waste compartment of the device of FIG. 1 along line A-A of FIG. 7C.

FIG. 8 is a sectional view of the cartridge of the device of FIG. 1.

FIG. 9 is a schematic diagram of an embodiment of the applicator head of the present device.

FIG. 10 is a schematic diagram of an embodiment of the applicator head for performing microdermabrasion of the skin of a user of the present device.

FIG. 11 is a schematic diagram of an embodiment of the applicator head for applying electricity to the skin of a user of the present device.

FIG. 12 is a schematic diagram of an embodiment of the applicator head for applying light energy to the skin of a user of the present device.

### SUMMARY

The present invention is a device for performing a treatment of the skin of a user of the device. The treatment involves the delivery of fluid to the user's skin surface, which can be accomplished with a device that includes a cartridge having a treatment fluid compartment for retaining a treatment fluid and a waste fluid storage compartment for receiving a waste fluid. The two compartments are in communication with a source of vacuum pressure, so that vacuum pressure from the outlet of a vacuum conduit of the waste fluid storage compartment pulls treatment fluid from an outlet of the treatment fluid compartment. A plunger in the proximal end of the treatment fluid compartment, in liquid-tight engagement from a proximal portion of the compartment is also moved distally at the same time.

The inlet of the vacuum conduit is positioned such that fluid in the waste fluid storage compartment will not reach the inlet when all the fluid from the treatment fluid compartment is contained in the waste fluid storage compartment. Preferably, the vacuum conduit is of sufficient length that fluid in the waste fluid storage compartment will not reach the inlet of the conduit when all the fluid from the treatment fluid compartment is contained in the waste fluid storage compartment. The use of a waste storage compartment having at least twice the volume of the treatment fluid compartment also can assist in preventing fluid flow into the vacuum conduit. The use of an absorbent material in the waste fluid storage compartment in an amount sufficient to absorb all the fluid from the treatment fluid compartment can also help to prevent the ingress of fluid into the vacuum conduit, particularly when the inlet of the vacuum conduit is surrounded by the absorbent material.

The device further includes an applicator tip or head having a fluid outlet in communication with the outlet opening of the treatment fluid compartment, a fluid inlet in communication with the fluid inlet of the waste fluid storage compartment, and a circumferential wall having a distal rim and surrounding the fluid outlet and the fluid inlet. The rim extends outwardly beyond the fluid outlet and the fluid inlet, so that when the rim contacts a user's skin, a seal can be formed around the rim to allow vacuum pressure from the source of vacuum pressure to pull the plunger of the treatment fluid compartment distally and withdraw treatment fluid from the treatment fluid compartment. After the treatment fluid is applied to the user's skin, the applied fluid is withdrawn through the fluid inlet of the applicator head and into the waste fluid storage compartment.

The treatment fluid can be any of a variety of fluids, but is preferably water or an aqueous solution. The treatment fluid can additionally comprise vitamins or hormones. In one embodiment, the treatment fluid can include an abrasive material, such as aluminum oxide, sodium bicarbonate, sodium chloride, silica, magnesium oxide, diamond, polyester, nylon.

In one embodiment, the applicator tip of the device can additionally comprise components for delivering microcurrent to the skin of a user. In this embodiment the applicator tip additionally includes an anode element having an upper surface and a cathode element having an upper surface. The upper surface of the anode and the upper surface of the cathode preferably extend outwardly beyond the distal ends of the fluid outlet and the fluid inlet of the applicator tip in order to make contact and be in electrical communication with a user's skin or at least with a fluid on the user's skin, thereby allowing the device to perform a microcurrent treatment. In one embodiment, the upper surface of the anode and the upper surface of the cathode extend outwardly to the same extent as the rim of the circumferential wall of the applicator tip. The microcurrent generator preferably delivers a current of between 1 and 1,000 microamperes, and more preferably of between 100 and 600 microamperes.

In other embodiments, the applicator tip can be provided with additional functionalities. For example, the rim of the circumferential wall of the applicator tip can comprise an abrasive material in order to perform a microdermabrasion treatment with the present device. Alternatively, the applicator tip can be provided with an ultrasound transducer, an LED light source, or a combination thereof.

### DESCRIPTION

The present device and method relate to the general field of skin care, and the manner in which a person cleans, exfoliates, rejuvenates and infuses treatment substances into the skin. The device described herein is a novel beautification device and method that utilizes a single hand held device to provide some of today's popular beautification modalities, including fluid assisted microdermabrasion and microcurrent treatments. In other embodiments, additional treatments such as light therapy and liquid infusion facial treatments can be provided.

Microdermabrasion is a treatment that involves the selective removal of the skin's surface cells. There are many iterations of this treatment, including the use of skin devices that combine an abrasive tip with a vacuum, for example devices that combine air pressure and a flow of powder, or vibrating and rotating skin brushes and pads. The benefits created are both in the increase reflectance of the skins surface (less visible lines and other flaws) and better absorption of topical treatment ingredients. Other skin therapies include: fluid-infusion facial, which uses a liquid or emulsion and typically air pressure to better deliver topical treatment ingredients into the skin; light therapy delivers specific wavelengths of visible and infrared light to the skin; vacuum therapies that use vacuum pressure; ultrasound; radio frequency and microwave energy therapy; massage therapy; and temperature (hot/cold) therapy.

There remains a need however for a device that can utilize fluid delivery and capture, and air pressure, to the skin and that can be used in combination with the foregoing treatments to create novel treatment methods. Further, there is a need for a device that is small, handheld, and that has detachable and interchangeable treatment heads, thus offering a plurality of treatment modalities from the same device.

### DEFINITIONS

As used herein, the following terms and variations thereof have the meanings given below, unless a different meaning is clearly intended by the context in which such term is used.

“Cartridge” refers to a component of the present device having a compartment for holding a powder or liquid and which can be inserted into a receptacle of the present device.

“Elongated” refers to a configuration or shape having a length which is longer than its width.

“Gasket” refers to a piece or ring of rubber or other material sealing the junction between two surfaces.

“Horizontal” refers to an orientation approximately parallel to (i.e., not substantially extending toward or away from) a surface or structure of the present device.

“Lower” refers to the relative position of a component in the present apparatus which is closer to or toward a surface or structure of the present device.

“Nozzle” refers to a projecting conduit with an opening for regulating and/or directing a flow of fluid.

“Outward” and “outwardly” mean in a direction away from a surface or structure of the present device.

“Plunger” refers to a piston-like reciprocating part moving within a conduit of a device.

“Tip” refers to the end of a structure or assembly.

“Tube”, “tubular” or “conduit” refers to a structure having a hollow, bore or through passage or other passageway, substantially aligned along a longitudinal axis of the structure and which may have various cross sections. Thus, these terms refer not only to a common tube having a circular cross section with a central opening, but also to other structures including those having square, elliptical or non-geometric and even irregular cross-sections, which include such a passageway.

“Vacuum line” as used herein is a tubular structure that interconnects other components of the system so as to form a vacuum pathway therebetween.

“Vacuum pump” is a device that removes gas molecules from a sealed volume in order to leave behind a partial vacuum.

“Upper” refers to the relative position of a component in the present apparatus which is further from or away from a surface or structure of the present device.

“Vertical” refers to an orientation extending toward or away from a support surface on which the present apparatus is supported when in use.

The term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

### Device Components

Handheld Unit

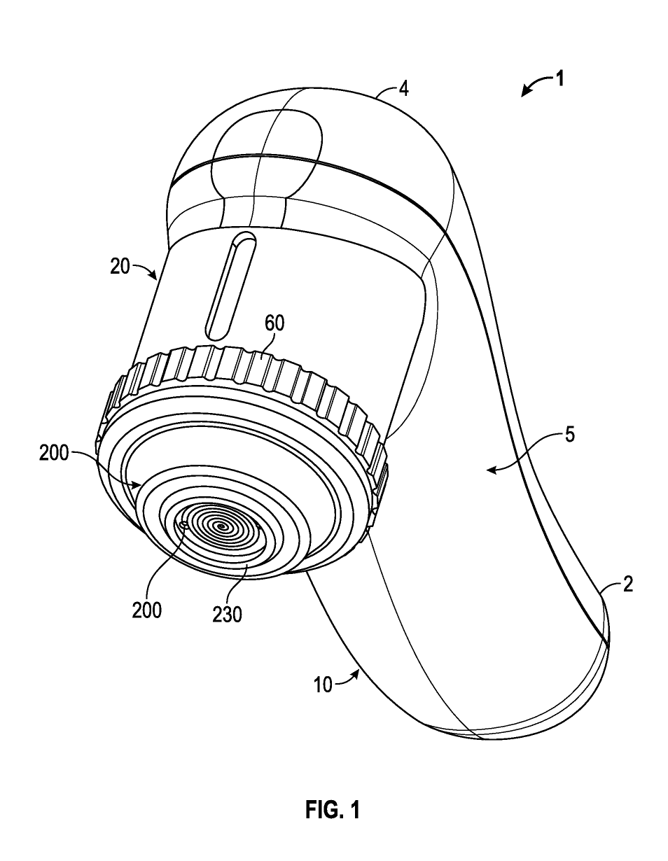

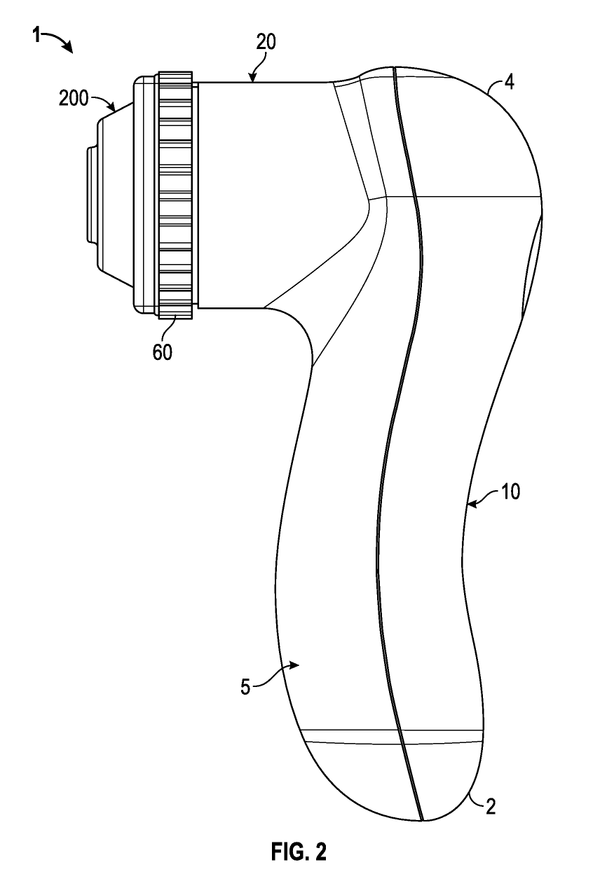

As seen for example in FIGS. 1-3, in a preferred embodiment the present device 1 comprises a handheld unit having a handle portion 10 for gripping the device 1 and a head portion 20 comprising the cartridge 100 and applicator tip 200 of the present device 1. The handle portion 10 generally extends from a proximal end 2 to a distal end 4 of the device, with the head portion 20 extending outwardly from the distal end 4.

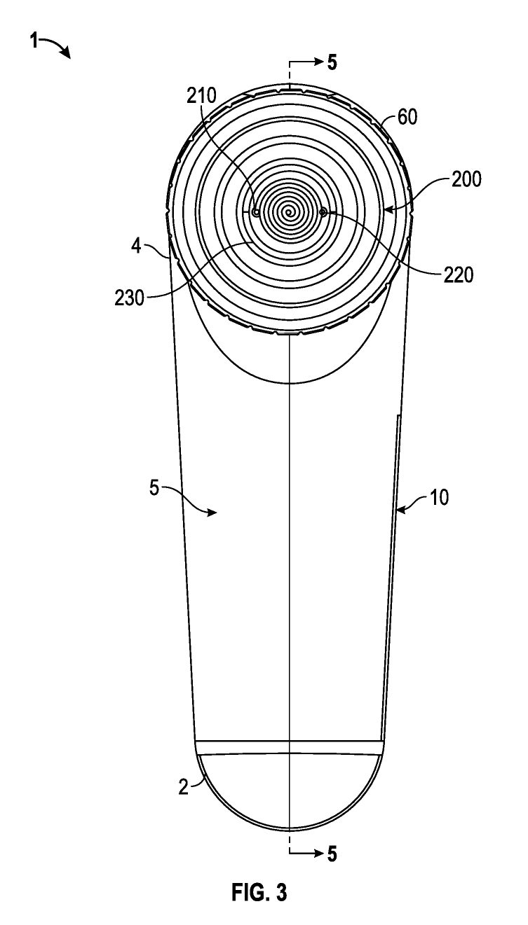

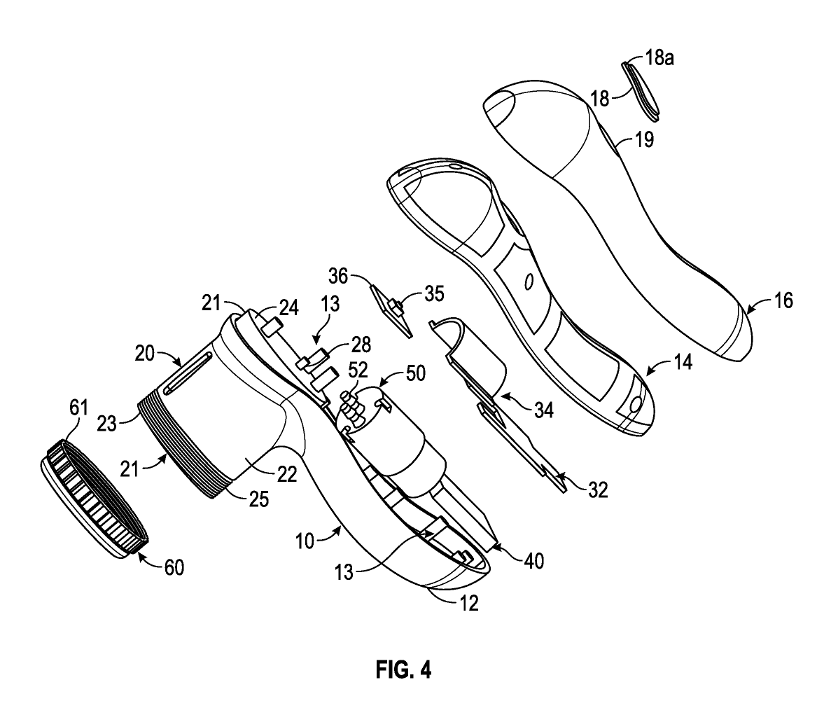

The components of the present device are illustrated in FIGS. 4-6. For manufacturing convenience, the housing 5 of the device 1 illustrated in FIGS. 1-6 comprises a front housing portion 12 and a rear housing portion 16 which join to form the housing 5 and enclose the internal components of the device. In the illustrated embodiment, the components contained in the handle portion 10 are retained in the housing by a cover 14, and the cover 14 is joined to the front housing portion 12 by fitting a shaft 11 extending away from the interior surface 15 of the cover 14 (i.e., inwardly) into the opening of a tubular receiving portion 13 extending away from the interior surface 17 of the front housing portion 12 (i.e., inwardly). The receiving portion 13 is sized to receive the shaft 11 and retain it with a friction fit, thereby retaining front housing portion 12 and cover 14 together. The shaft 11 can alternatively be retained on the rear housing portion 16, or the receiving portion 13 can alternatively be positioned on the rear housing portion 14 and the shaft 11 on the cover 14 or the front housing portion 12. Other ways to join front housing portion 12 and rear housing portion 14 known to the art can also be used. The rear housing portion 16 is placed over the cover 14, and can be retained on the cover by means of an adhesive, a friction fit, or in other ways known to the art. The housing 5 is preferably formed from a rigid material such as a rigid plastic polymer.

Contained within the housing 5 of the handle portion 10 in this embodiment is a source of vacuum pressure, such as a vacuum pump 50, which can be an electro-mechanical pressure generator, such as an AC or a DC air pump. The vacuum source can generate between 1 and 14 psi of force, for example. Alternatively, the present device can include a pressure holding chamber or chambers within the device, in which case the pressure generating component would be external to the device. Although vacuum pressure is used in the present device to deliver and remove a treatment fluid, in an alternative embodiment the vacuum pressure can be applied to a user's skin for its own therapeutic effects, without the application of a fluid. The vacuum pump 50 is preferably secured within the housing 5 by a vacuum retaining portion 34, which has an interior surface that contacts and engages the exterior surface of the vacuum pump 50 and has outwardly extending flanges which secure the vacuum retaining portion 34 to the interior walls of the housing 5.

The handle portion 10 can also include a battery 40 for powering the vacuum pump 50. In the illustrated embodiment, the battery 40 is positioned below the vacuum pump 50, although other arrangements are also possible. The battery is preferably rechargeable, in which case it can be placed in electrical communication with a connector for connection to an electrical cord, in order to place the battery 40 in electrical communication with an electrical outlet or other source of power. The present device 1 can alternatively be powered solely by connection to an electrical outlet via an electrical cord. In another embodiment, the battery is charged by an inductive coupling to a charging station, as is known to the art. The battery 40 can also be a single-use battery, in which case the rear housing portion 16 and cover 14 (if present) must be reversibly secured to the front housing portion 12 of the device 1. In embodiments in which a rechargeable battery is used, the battery 40 is preferably secured within the housing 5 by a battery plate 32, which contacts a rear surface of the battery 40 and includes outwardly extending flanges which secure the battery plate 32 to the interior walls of the housing 5.

Power to the device 1 can be controlled with an on/off switch 36 which is preferably actuated by depressing a button 18. The button 18 is retained within an opening 19 of the rear portion 16 of the housing 5, preferably in a distal end 4 of the device, and includes a circumferential flange 18a maintained within the interior of the housing 5 in order to secure the button 18 to the device. The button 18 is preferably formed from an elastomeric or other flexible material, and actuates the switch when a user applies pressure to the exterior surface 37 of the button 18. The pressure is relayed to the distal end 35 of an electrical switch 36 by a post 37 extending inwardly from the rear surface of the button 18.

In a preferred embodiment, fluid used in the treatments accomplished with the present device 1 is contained in a cartridge 100, and the cartridge 100 is retained in a head portion 20 of the device. The head portion 20 includes an opening 21 in a distal portion 23 of the head portion 20 for receiving such a cartridge 100, and further includes circumferential walls 22 having an interior surface 26 for engaging the cartridge 100. Grooves 25 are preferably incorporated into the exterior surface of the distal end 23 of the head portion 20 and mate with threads 61 on an interior surface of a tip cover 60, which is used to secure attachment tips 200 to the distal end 23 of the head portion 20. The head portion 20 also preferably includes a rear wall 24 in a proximal end 21 of the head portion 20 for better securing the cartridge 100. The rear wall 24 further preferably includes a vacuum line connector 28 for securing a vacuum line (not shown) between a vacuum port 52 of the vacuum pump 50 and the vacuum line connector 28.

Cartridge

The head portion 20 in the illustrated embodiments retains within it a treatment fluid compartment or reservoir 120 and a waste fluid storage compartment 160, which are preferably provided together as a unitary cartridge 100. In the illustrated embodiments, the treatment fluid compartment 120 comprises a proximal end 121, a distal end 123, an interior compartment 125 for retaining treatment fluid, an outlet 128, and a plunger 130. The distal face 132 of the plunger is in communication with the interior of the compartment 125 (and the treatment fluid, when present), and outer edges 133 of the plunger 130 seal around the interior compartment 125 so that fluid can be retained within the compartment 125. The plunger 130 is preferably formed from an elastomeric or other flexible material for this reason. The plunger 130 is also moveable, so that as a treatment fluid is withdrawn from the compartment 125 in response to vacuum pressure from the vacuum pump 50, the plunger 130 is moved distally toward the distal end 123 of the treatment fluid compartment 120. A valve (not shown), which can be electrical or mechanical, can be positioned either in the outlet 128 or downstream thereof in order to prevent used fluid from returning to the compartment 125.

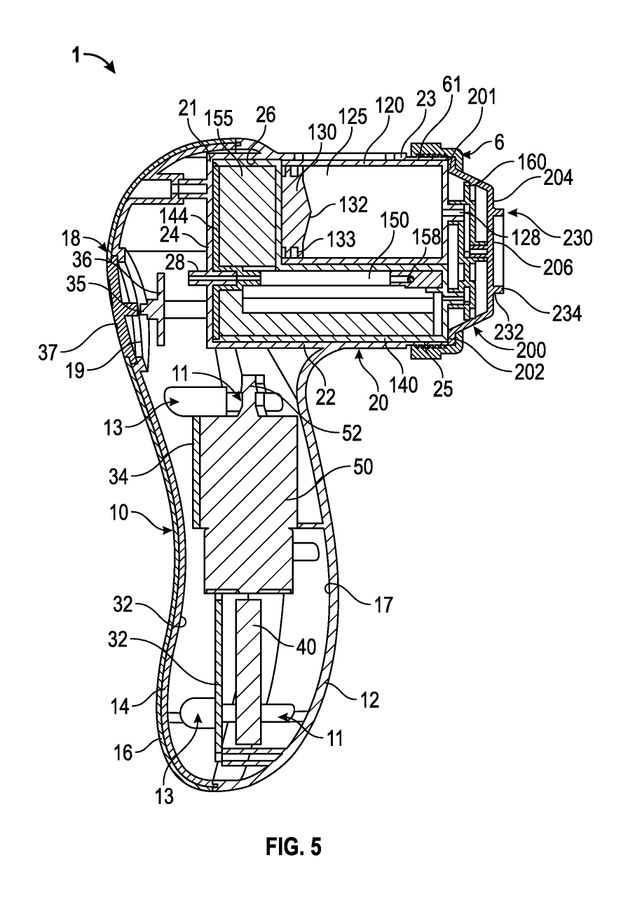

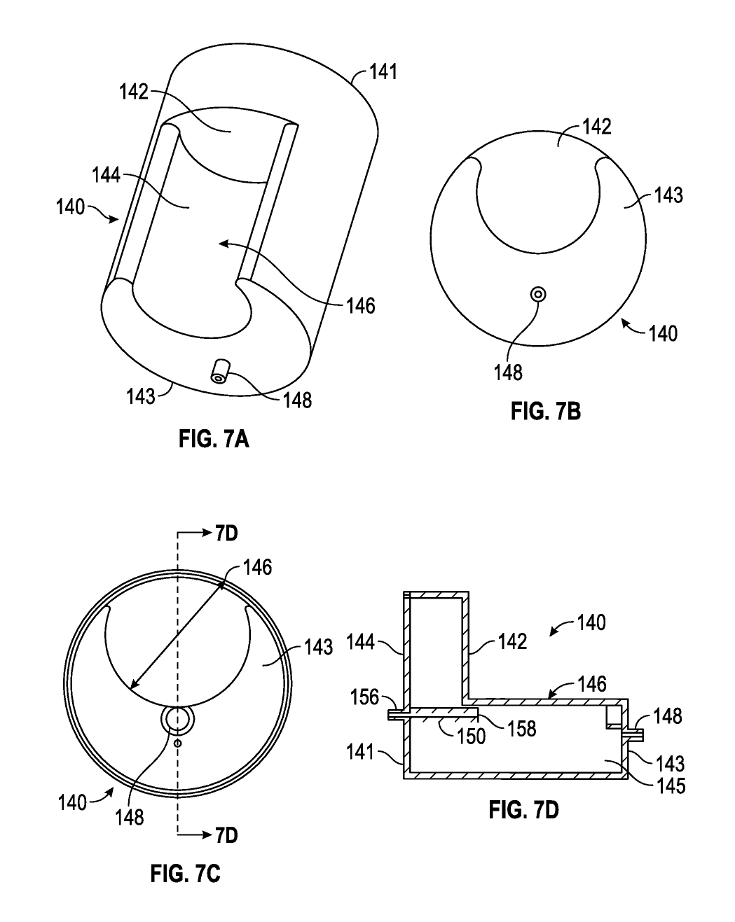

The waste fluid storage compartment 140 similarly comprises a proximal end 141, a distal end 143, an interior compartment 145 for receiving used treatment fluid, and an inlet 148. In the embodiment of FIGS. 5-7, the waste fluid compartment 140 includes a cradle or receptacle 146 sized to receive and retain the treatment fluid compartment 120, such that the proximal end 121 of the treatment fluid compartment 120 is adjacent to a rear wall 142 of the receptacle 146. In preferred embodiments, the volume of the interior compartment 145 of the waste fluid storage compartment 140 will be larger than that of the interior compartment 124 of the treatment fluid compartment 120. The treatment fluid compartment 120 can be retained within the receptacle 146 by a friction fit, an adhesive, or in other ways known to the art. A valve (not shown), which can be electrical or mechanical, can be positioned either in the inlet 148 or upstream thereof in order to prevent used fluid from leaving the compartment 145.

The fluid inlet 148 into the waste fluid storage compartment 140 carries used treatment fluid and skin fragments to the interior compartment 145. The inlet 148 preferably directs fluid entering the compartment against an interior wall or otherwise away from an outlet leading to the vacuum conduit inlet 158.

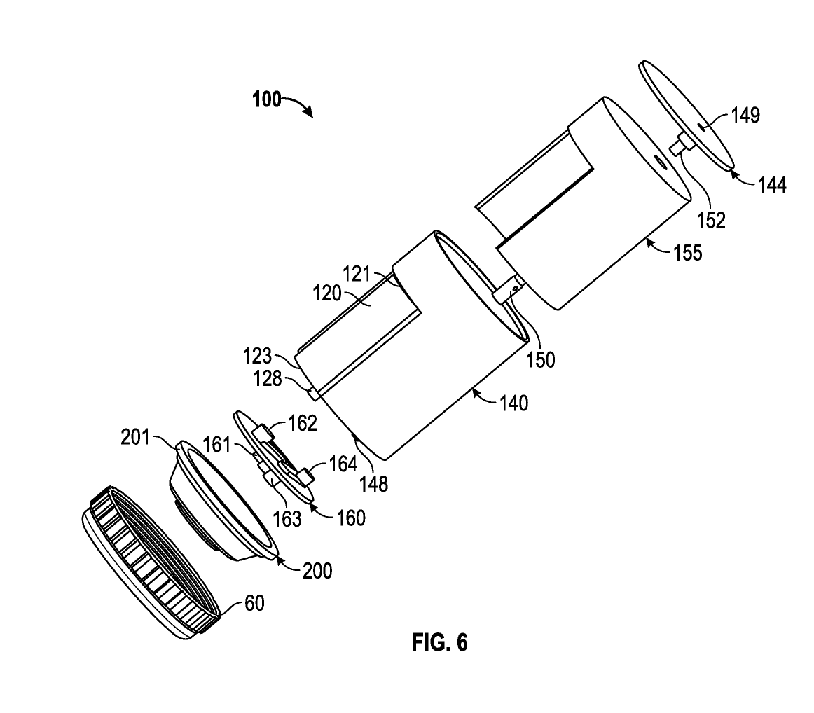

The waste storage compartment 140 further comprises a vacuum conduit 150. The vacuum conduit 150 includes an inlet 158, from which air is withdrawn from the waste fluid storage compartment 140, and an outlet 156. In the embodiment of FIGS. 5 and 6, the outlet connects to the distal end of a connector 152 in the rear wall 144 of the waste storage compartment 140, and the proximal end of the connector 152 then connects to the vacuum line connector 28 in the rear wall 24 of the head portion 20, in order to place the vacuum conduit 150 in communication with the vacuum pressure of the vacuum pump 50. Alternatively, the rear wall 24 of the head portion 20 can simply comprise an opening, and the outlet 156 of the vacuum conduit 150 can connect directly to the vacuum line.

The inlet 158 of the vacuum conduit 150 is preferably positioned and/or has a sufficient length such that fluid in the waste fluid storage compartment 140 will not reach the inlet 158 when all the fluid from the treatment fluid compartment is contained in the waste fluid storage compartment 140. This can be accomplished in part by providing an interior compartment 145 of the waste fluid storage compartment 140 that has a volume greater than that of the interior compartment 125 of the treatment fluid compartment, and preferably has a volume at least twice as great as the interior compartment 145. In this case, when the vacuum conduit extends into an interior portion of the interior compartment 145, the fluid level of the waste fluid in the compartment will always remain below the opening of the inlet 158.

The waste fluid storage compartment 140 can also preferably comprise an absorbent material 155 within the interior compartment 145. The absorbent material can be any material capable of absorbing and retaining some or all of the used treatment fluid entering the interior compartment 145. The absorbent material can, for example, be cotton or an absorbent polymer such as that used in disposable diapers. In one preferred embodiment, sufficient absorbent material 155 can be used in the waste fluid storage compartment 140 that all of the fluid retained by the treatment fluid compartment 120 can be absorbed. In this embodiment the vacuum conduit 150 can be positioned in any convenient location within the interior of the interior compartment 145 of the waste fluid storage compartment 140, as long as the absorbent material is positioned between the inlet 148 of the compartment 145 and the inlet 158 of the vacuum conduit 150.

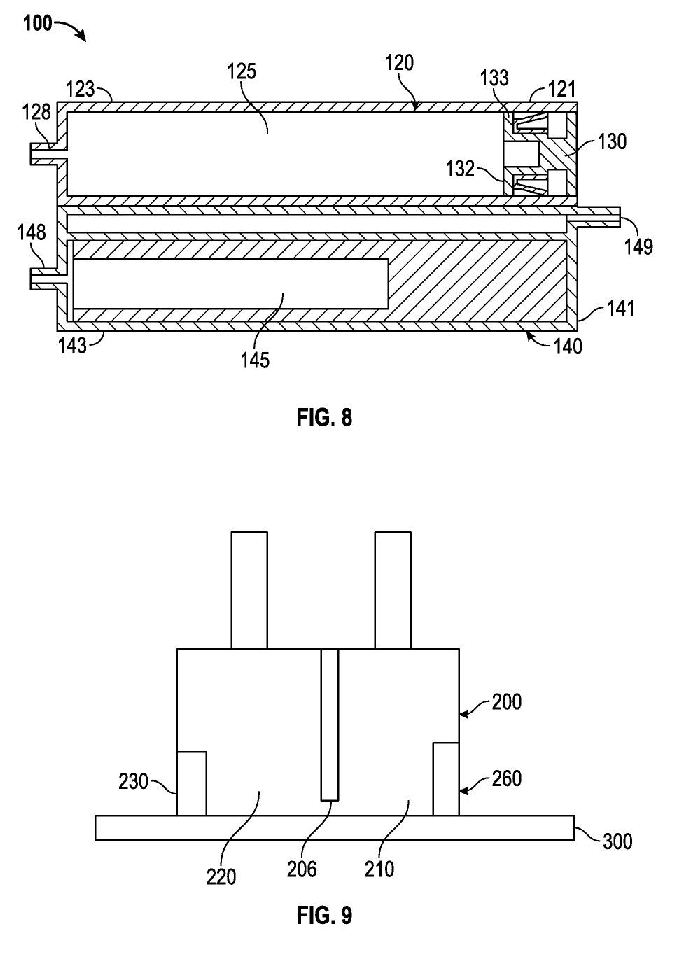

The fluid storage compartments of the present device are preferably housed within a single cartridge, which can be disposable or refillable. In the illustrated embodiments, the cartridge 100 is inserted through the opening 21 in the head portion 20 such that the rear wall 144 of the waste fluid storage compartment 140 is placed adjacent to, and preferably in contact with, the rear wall 24 of the head portion 20. In alternative embodiments, such as that shown in FIG. 8, the treatment fluid compartment 120 and the waste fluid storage compartment 140 can be separate units, and can be placed separately into the head portion 20, either into a common compartment or into separate compartments. A light indicator or some other visual indicator can be used to show when the fluid storage compartments needs to be changed, emptied or refilled.

### Applicator Tip

The present device 1 further includes an applicator head or tip 200 having a proximal end 202 at or adjacent to the distal end 23 of the head portion 20 of the device. In the embodiments shown in FIGS. 1-6, the applicator tip 200 is secured to the head portion 20 by a tip cover 60 having threads 61 on an interior surface that mate with grooves 25 on the exterior surface of the distal end 23 of the head portion 20. The interior surface of the tip cover 60 contacts the distal surface of a circumferential flange 201 of the applicator tip 200, while the proximal surface of the flange 201 contacts the distal end of either the head portion 20 or the cartridge 100, and the tip cover 60 thereby holds the applicator tip 200 in place by a friction fit. Other ways to attach the applicator tip to the distal end of the head portion 20 and/or the cartridge 100 can also be used.

The applicator tip 200 further includes a fluid outlet 210 and a fluid inlet 220 which open at a distal end at or adjacent to a distal surface 206 of the applicator tip 200. The fluid outlet 210 is in fluid communication with the outlet opening 128 of the treatment fluid compartment 120, and the fluid inlet 220 of the applicator tip 200 is in fluid communication with the fluid inlet 148 of the waste fluid storage compartment 140. Communication between these components can be accomplished through the use of conduits or in other ways known to the art. In the embodiment illustrated in FIGS. 5 and 6, the fluid outlet 210 and fluid inlet 220 of the applicator tip are connected to the outlet opening 128 of the treatment fluid compartment 120 and with the fluid inlet 148 of the waste fluid storage compartment 140, respectively, by a fluid connector 160 having inlet conduits 162 and 164 for connection at a proximal end to the outlet opening 128 of the treatment fluid compartment 120 and to the fluid inlet 148 of the waste fluid storage compartment 140, respectively. The distal end 161 of fluid conduit 162 connects with the outlet 210 of the applicator tip 200, while the distal end 163 of fluid conduit 164 connects with the inlet 220.

The applicator tip 200 further includes a circumferential wall 230 which extends outwardly from a proximal end 232 at the surface 206 of the applicator tip 200 to a rim 234 at its distal end. The wall 230 surrounds the fluid outlet 210 and the fluid inlet 220. Preferably, the rim 234 is approximately planar, so that when the rim 234 contacts a user's skin, a seal can be formed around the rim. When a seal or an approximate seal is formed, vacuum pressure from the vacuum pump 50 is able to pull the treatment fluid from the treatment fluid compartment 120 as well as pulling the plunger 130 of the treatment fluid compartment 120 and thereby withdraw treatment fluid from the fluid outlet 210 of the applicator tip. The vacuum pressure also draws the fluid applied to the user's skin, mixed with dead skin cells and other debris, into the fluid inlet 220 of the applicator tip 200, and from there into the waste fluid storage compartment 140.

Alternative Applicator Tips

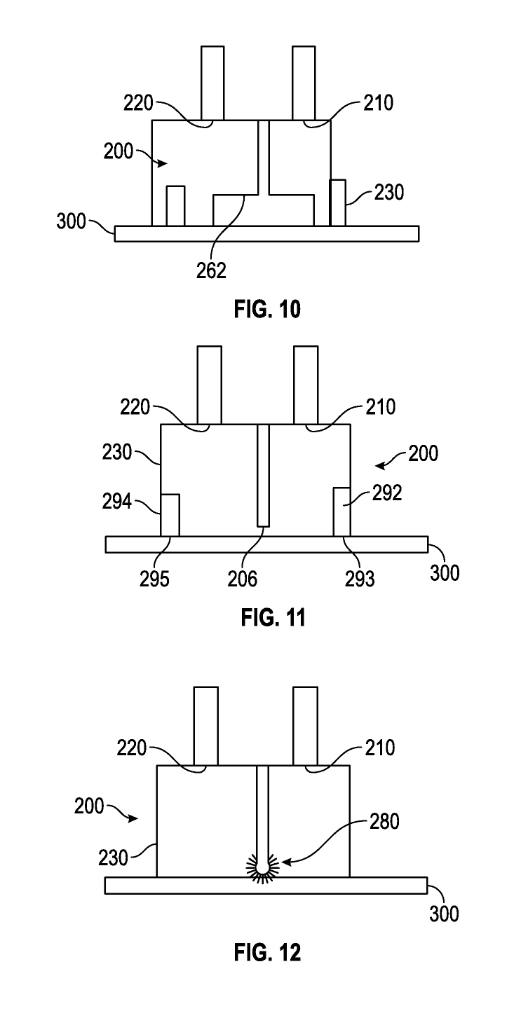

The applicator tip 200 can, in addition to delivering a fluid to a user's skin, be provided with additional functionalities. In one embodiment, an abrasive treatment head can be placed in contact with a user's skin 300 when the rim 234 is in contact with the user's skin, in order to exfoliate the skin. In the embodiment shown in FIG. 9, the rim 234 is provided with an abrasive material 260 and/or with one or more sharp edges, so that the rim 234 is thereby provided an abrasive surface. In another embodiment, shown in FIG. 10, a piece of abrasive material 262 can be provided within the rim 234, as shown in FIG. 10, as long as fluid communication is provided between the outlet 210 and the inlet 220 of the applicator tip 200. By combining an abrasive tip with a vacuum source, visible lines and other flaws in a user's skin can be reduced, and better absorption of topical treatment ingredients can be achieved. The abrasive surface can also be made to vibrate and or rotate.

In another embodiment, an ultrasound wave generator can be provided on the distal end of the applicator tip 200 in order to deliver waves at ultrasonic speeds to the skin 300 of a user of the device 1. These waves can be, but are not restricted to be, in the range of 1 Mhz to 6 Mhz. For example, a treatment head comprising an ultrasonic transducer can be provided within the wall 230 of the applicator tip 200.

In yet another embodiment, shown in FIG. 11, the applicator tip 200 can comprise components for delivering microcurrent to the skin of a user. In this embodiment, the applicator tip additionally includes an anode element 292 having an upper surface 293 and a cathode element 294 having an upper surface 295. The upper surface 293 of the anode 292 and the upper surface 295 of the cathode 294 preferably extend outwardly beyond the distal ends of the fluid outlet and the fluid inlet of the applicator tip in order to make contact and be in electrical communication with a user's skin or at least with a fluid on the user's skin, thereby allowing the device to perform a microcurrent treatment. In one embodiment, the upper surface 293 of the anode 292 and the upper surface 295 of the cathode 294 extend outwardly to the same extent as the rim 234 of the circumferential wall of the applicator tip 200. The microcurrent generator preferably delivers a current of between 1 and 1,000 microamperes, more preferably of between 100 and 600 microamperes, and at frequencies of between about 3 and 10 hertz. Polarity altering from positive to negative can also be provided for a duration ranging from about 1 to about 4 seconds for each of the polarities. The anode 292 and cathode 294 are formed from a conductive material such as a metal and are in electrical communication with a microcurrent generator located either in the housing 5 or in the cartridge 100, as well as with the battery 40.

In a further embodiment, shown in FIG. 12, the applicator tip 200 can be provided with a source of light energy 280, such as an LED of flash lamp, in order to deliver light to the skin, for example visible or infrared light. The LED or other source can be provided either within the wall 230 at the distal end of the applicator tip 200 or can be provided outside the walls. The light source is in electrical communication with the battery 40, and in some embodiments can be in electrical communication with circuits for controlling the output of the light source 280, for example to provide pulsating light and/or a steady stream of light.

Another embodiment of the present device can incorporate a needle, a combination of needles, or another means for creating microperforations in the skin, in order to deliver radio frequency waves to the skin. The radio waves can be communicated to the skin through an independent element or through microneedles or the microperforation source. Electrical current can also be provided to the skin through the microneedles.

When the present device is designed to be operable with alternative applicator tips 200, it can be fitted with a circuit and use software and/or firmware to recognize the particular applicator tip 200 that is fitted on the device. The circuit will also have the ability to control the relevant functions of the applicator tips.

Materials

Any of a variety of treatment fluids can be used in the present device. In one embodiment, the treatment fluid is water or an aqueous solution. Such a solution can comprise vitamins, hormones, a peeling agent, a nourishing agent, a medication, botanicals, a plumping agent and other compositions known for topical use, as well as combinations thereof. The treatment fluid can alternatively be a serum used for the treatment of skin.

In an alternative embodiment, the treatment fluid can be used for microdermabrasion and can comprise a crystalline or other fine particulate material. A variety of abrasive materials suitable for topical use can be used, such as aluminum oxide, sodium bicarbonate, sodium chloride, silica, magnesium oxide, salicylic acid, diamond, polyester, nylon, or organic grains made from plant sources such as trees, straw, reeds, maize, sunflowers, plants, or cane sugar. The particulates are preferably between about 50 and 50 micrometers in size, and more preferably are about 100 micrometers in size. A variety of fluids known to the art can be used as the carrier fluid for the particulate material, with the choice of fluid dependent in part on the nature of the abrasive particulate being used as well as on the choice of other cosmetic or therapeutic qualities sought to be imparted by the present treatment. When water soluble abrasive materials are used, for example, a non-aqueous carrier fluid should be selected.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure.

Recitation of value ranges herein is merely intended to serve as a shorthand method for referring individually to each separate value falling within the range. Unless otherwise indicated herein, each individual value is incorporated into the specification as if it were individually recited herein. All references cited herein are incorporated by reference in their entirety.

What is claimed is:

1. A device for performing microdermabrasion, comprising:
(a) a cartridge comprising:
(i) a treatment fluid compartment for retaining a treatment fluid, the compartment having a proximal end, a distal end, and a cylindrical inner wall, wherein the proximal end further comprises a plunger sealing the proximal end in liquid-tight engagement from a proximal portion of the compartment, and wherein the distal end comprises an outlet opening;
(ii) a waste fluid storage compartment for receiving a waste fluid and having a proximal end, a distal end, and a fluid inlet, wherein the waste fluid storage compartment has a larger interior volume than the treatment fluid compartment; and
(iii) a vacuum conduit within the waste fluid storage compartment having an inlet in communication with the interior of the waste fluid storage compartment and an outlet outside the waste fluid storage compartment, wherein the inlet of the vacuum conduit is positioned such that fluid in the waste fluid storage compartment will not reach the inlet when all the fluid from the treatment fluid compartment is contained in the waste fluid storage compartment;
(b) a source of vacuum pressure in communication with the outlet of the vacuum conduit of the waste fluid storage compartment;
(c) an applicator head having:
(i) a fluid outlet having a distal end, the fluid outlet being in fluid communication with the outlet opening of the treatment fluid compartment;
(ii) a fluid inlet having a distal end, the fluid inlet being in fluid communication with the fluid inlet of the waste fluid storage compartment; and
(iii) a circumferential wall having a distal rim and surrounding the fluid outlet and the fluid inlet, wherein the rim extends outwardly beyond the fluid outlet and the fluid inlet,
wherein when the rim of the circumferential wall contacts a user's skin, a seal can be formed around the rim to allow vacuum pressure from the source of vacuum pressure to pull the plunger of the treatment fluid compartment distally and withdraw treatment fluid from the treatment fluid compartment through the fluid outlet of the applicator head, following which fluid applied to the user's skin is withdrawn through the fluid inlet of the applicator head and into the waste fluid storage compartment.

2. The device of claim 1, further comprising an absorbent material in the waste fluid storage compartment.

3. The device of claim 2, wherein the amount of the absorbent material is sufficient to absorb all the fluid from the treatment fluid compartment, and wherein the inlet of the vacuum conduit is surrounded by the absorbent material.

4. The device of claim 1, wherein the waste fluid storage compartment has at least twice the volume of the treatment fluid compartment.

5. The device of claim 1, wherein the vacuum conduit is of sufficient length that fluid in the waste fluid storage compartment will not reach the inlet of the conduit when all the fluid from the treatment fluid compartment is contained in the waste fluid storage compartment.

6. The device of claim 1, further comprising treatment fluid in the treatment fluid compartment.

7. The device of claim 6, wherein the treatment fluid is an aqueous solution.

8. The device of claim 7, wherein the treatment fluid additionally comprises vitamins or hormones.

9. The device of claim 6, wherein the treatment fluid comprises an abrasive material selected from the group consisting of aluminum oxide, sodium bicarbonate, sodium chloride, silica, magnesium oxide, diamond, polyester, nylon.

10. The device of claim 1, wherein the device additionally comprises:
an anode having an upper surface; and
a cathode having an upper surface,
wherein the upper surface of the anode and the upper surface of the cathode extend outwardly beyond the distal ends of the fluid outlet and the fluid inlet and are in electrical communication with the treatment fluid applied to the user's skin, thereby allowing the device to perform a microcurrent treatment.

11. The device of claim 10, wherein the upper surface of the anode and the upper surface of the cathode extend outwardly beyond the distal end of the fluid outlet and the distal end of the fluid inlet to the same extent as the rim of the circumferential wall of the applicator head.

12. The device of claim 10, wherein a current of between 10 and 1,000 microamperes can be applied between the anode and the cathode.

13. The device of claim 12, wherein a current of between 100 and 600 microamperes can be applied between the anode and the cathode.

14. The device of claim 1, wherein the rim of the circumferential wall comprises an abrasive material.
